(19) United States
(12) Patent Application Publication
(10) Pub. No.: US 2025/0276007 A1
(43) Pub. Date: Sep. 4, 2025

(54) CARBAMOYLPHENYLBORONIC ACID-CONTAINING ACRYLIC MONOMERS AND USES THEREOF

(71) Applicant: Alcon Inc., Fribourg (CH)

(72) Inventors: Frank Chang, Cumming, GA (US); Troy Vernon Holland, Suwanee, GA (US); Adam K. Sniady, Lilburn, GA (US)

(21) Appl. No.: 19/068,731

(22) Filed: Mar. 3, 2025

Related U.S. Application Data
(60) Provisional application No. 63/560,858, filed on Mar. 4, 2024.
(60) Provisional application No. 63/560,860, filed on Mar. 4, 2024.

(51) Int. Cl. A61K 31/785; A61P 27/04; C08L 85/02; C08L 85/04

(52) U.S. Cl. CPC A61K 31/785; A61P 27/04; C08L 85/02; C08L 85/04

(57) ABSTRACT

The invention is generally related to an acrylamido monomer having one (carbamoylphenyl)boronic acid and a pKa of from about 6.4 to about 7.6 and to a water-soluble hydrophilic copolymers each comprising repeating units of such an acrylamido monomer.

This application claims the benefits under 35 USC § 119 (e) of U.S. provisional application Nos. 63/560,858 and 63/560,860 both filed on 4 Mar. 2024, herein incorporated by reference in their entireties.

The present invention generally relates to a class of acrylamido monomers each of which comprises a (carbamoylphenyl)boronic acid and has a pKa of about 6.4 to about 7.8.

### BACKGROUND

It has been known that phenylboronic acid derivatives can be used as receptors and sensors for carbohydrates and small molecules, largely due to their capability of forming cyclic boronate esters with cis-diols (see, e.g., S. D. Bull, et al., Acc. Chem. Res. 2013, 46 (2), 312-326; J. A. Peters, Coord. Chem. Rev. 2014, 268, 1-22). In recent years, aryl boronic acid-containing vinylic monomers have been developed and used to prepare aryl boronic acid-containing polymers (see, e.g., T. Konno & K. Ishihara, Biomater. 2007, 28, 1770-1777; Z. Liu & H. He, Acc. Chem. Rev. 2017, 50, 2185-2193; W. L. A. Brook & B. S. Summerlin, Chem. Rev. 2016, 116, 1375-1397; Z. Huang, et al., Biomater. Sci. 2018, 6, 2487-2495; J. N. Cambre & B. S. Sumerlin, Polymer 2011, 52, 4631-4643; Z. H. Farooqi, et al., Macromol. Chem. Phys. 2011, 212, 1510-1514; S. Roy, et al., Designed Monomers and Polymers 2009, 12, 483-495; D. Zhang & R. Pelton, Langmuir 2012, 28, 3112-3119; G. Vancoillie & R. Hoogenboom, Polym. Chem. 2016, 7, 5484-5495).

By having aryl boronic acid moieties, such aryl boronic acid-containing polymers can be complexed with mucin, especially epithelial mucin. As such, it is reported that they can be used in ophthalmic devices and ophthalmic compositions (see, e.g., U.S. Pat. Nos. 7,988,988, 8,419,792, 8,534,031, 8,631,631, 8,668,736, 6,596,267, 8,802,075, 9,244,196, 10,962,803, 11,439,661; U.S. Pat. Appl. Pub. No. 2021-0077385). However, the aryl boronic acid vinylic monomers used in preparation of aryl boronic acid-containing polymers may have a pKa too high for ensuring those aryl boronic acid-containing polymer to have a strong interaction with mucin.

Therefore, there are still needs for aryl boronic acid-containing vinylic monomers having a low pKa.

### SUMMARY

In one aspect, the invention provides an acrylamido monomer comprising an acrylamido group and a (carbamoylphenyl)boronic acid that is covalent linked to the acrylamido group through a linkage, wherein the acrylamido monomer has a pKa of from about 6.4 to about 7.6.

In another aspect, the invention provides A water-soluble hydrophilic copolymer, comprising: (a) repeating monomeric units of at least one acrylamido monomer of the invention; (b) repeating units of at least one hydrophilic vinylic monomer; and optionally (c) repeating units of at least one hydrophilic vinylic crosslinker, provided that the components (a) and (b) are present in the water-soluble hydrophilic copolymer in a total amount of at least 90% by mole.

In a further aspect, the invention provides an ophthalmic composition comprising: a water-soluble hydrophilic copolymer of the invention.

### DETAILED DESCRIPTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art.

“About” as used herein means that a number referred to as “about” comprises the recited number plus or minus 1-10% of that recited number.

“Optional” or “optionally” means that the subsequently described event or circumstance can or cannot occur, and that the description includes instances where the event or circumstance occurs and instances where it does not.

A “vinylic monomer” refers to a compound that has one sole ethylenically-unsaturated group.

The term “soluble”, in reference to a compound or material in a solvent, means that the compound or material can be dissolved in the solvent to give a solution with a concentration of at least about 0.1% by weight at room temperature (i.e., from about 20° C. to about 30° C.).

The term “insoluble”, in reference to a compound or material in a solvent, means that the compound or material can be dissolved in the solvent to give a solution with a concentration of less than 0.005% by weight at room temperature (as defined above).

The term “ethylenically unsaturated group” is employed herein in a broad sense and is intended to encompass any groups containing at least one >C═C< group. Exemplary ethylenically unsaturated groups include without limitation (meth)acryloyl

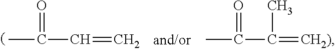

allyl, vinyl (—CH═CH₂) 1-methylethenyl

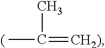

styrenyl, or the likes.

The term “(meth)acrylamide” refers to methacrylamide and/or acrylamide.

The term “(meth)acrylate” refers to methacrylate and/or acrylate.

A “hydrophilic vinylic monomer”, as used herein, refers to a vinylic monomer which can be polymerized to form a homopolymer that is water-soluble or can absorb at least 10 percent by weight of water.

A “hydrophobic vinylic monomer” refers to a vinylic monomer which can be polymerized to form a homopolymer that is insoluble in water and can absorb less than 10 percent by weight of water.

An “acrylic monomer” refers to a vinylic monomer having one sole (meth)acryloyl group.

An “acrylamido monomer” refers to a vinylic monomer having one sole (meth)acrylamido group

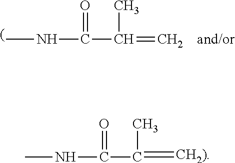

As used in this application, the term “vinylic crosslinker” refers to a compound having at least two ethylenically unsaturated groups. A “vinylic crosslinking agent” refers to a vinylic crosslinker having a molecular weight of about 700 Daltons or less.

As used in this application, the term “polymer” means a material formed by polymerizing/crosslinking one or more monomers or macromers or prepolymers.

A “vinyl-based copolymer” refers to a copolymer of at least two different vinylic monomers.

As used in this application, the term “molecular weight” of a polymeric material (including monomeric or macromeric materials) refers to the weight-average molecular weight unless otherwise specifically noted or unless testing conditions indicate otherwise.

The term “alkyl” refers to a monovalent radical obtained by removing a hydrogen atom from a linear or branched alkane compound. An alkyl group (radical) forms one bond with one other group in an organic compound.

The term “alkylene divalent radical” or “alkylene diradical” or “alkyl diradical” interchangeably refers to a divalent radical obtained by removing one hydrogen atom from an alkyl. An alkylene divalent group forms two bonds with other groups in an organic compound.

The term “alkyl triradical” refers to a trivalent radical obtained by removing two hydrogen atoms from an alkyl. An alkyl triradical forms three bonds with other groups in an organic compound.

The term “alkoxy” or “alkoxyl” refers to a monovalent radical obtained by removing the hydrogen atom from the hydroxyl group of a linear or branched alkyl alcohol. An alkoxy group (radical) forms one bond with one other group in an organic compound.

In this application, the term “substituted” in reference to an alkyl diradical or an alkyl radical means that the alkyl diradical or the alkyl radical comprises at least one substituent which replaces one hydrogen atom of the alkyl diradical or the alkyl radical and is selected from the group consisting of hydroxy (—OH), carboxy (—COOH), —NH₂, sulfhydryl (—SH), C₁-C₄ alkyl, C₁-C₄ alkoxy, C₁-C₄ alkylthio (alkyl sulfide), C₁-C₄ acylamino, C₁-C₄ alkylamino, di-C₁-C₄ alkylamino, halogen atom (Br or Cl), and combinations thereof.

In this application, an “aryl boronic acid-containing vinylic monomer” refers to a vinylic monomer which comprises one sole aryl boronic acid group linked to its sole ethylenically unsaturated group through one linkage.

In this application, an “arylborono” group refers to a monovalent radical of

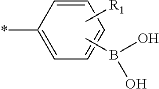

in which R₁ is a monovalent radical (preferably H, NO₂, F, Cl, Br, CF₃, CH₂OH, or CH₂NR^{o}R^{o}′ in which R^{o} and R^{o}′ independent of each other are H or C₁-C₄ alkyl). It is understood that where R₁ is CH₂OH, or CH₂NR^{o}R^{o}′, it is at the ortho-position of the boronic acid and can form intramolecular B—O or B—N coordination to lower the pKa of the boronic acid.

In this application, an “phosphorylcholine-containing vinylic monomer” refers to a vinylic monomer which comprises one sole arylborono group linked to its sole ethylenically unsaturated group through one linkage.

As used in this application, the term “phosphorylcholine” refers to a monovalent zwitterionic group of

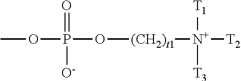

in which t1 is an integer of 1 to 5 and T₁, T₂ and T₃ independently of one another are C₁-C₈ alkyl or C₁-C₈ hydroxyalkyl.

The term “azlactone” refers to a mono-valent radical of

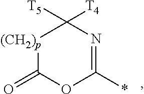

in which p is 0 or 1; T₄ and T₅ independently of each other is an alkyl group having 1 to 14 carbon atoms, a cycloalkyl group having 3 to 14 carbon atoms, an aryl group having 5 to 12 ring atoms, an arenyl group having 6 to 26 carbon and 0 to 3 sulfur, nitrogen and/or oxygen atoms, or T₄ and T₅ taken together with the carbon to which they are joined can form a carbocyclic ring containing 5 to 8 ring atoms.

In this application, a “(carbamoylphenyl)boronic acid” refers to a monovalent radical of

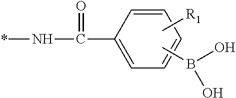

in which R₁ is a monovalent radical (preferably H, NO₂, F, Cl, Br, CF₃, CH₂OH, or CH₂NR^{o}R^{o}′ in which R^{o} and R^{o}′ independent of each other are H or C₁-C₄ alkyl). It is understood that where R₁ is CH₂OH, or CH₂NR^{o}R^{o}′, it is at the ortho-position of the boronic acid and can form intramolecular B—O or B—N coordination to lower the pKa of the boronic acid.

A “mucoadhesive polymer” refers to a polymer capable of being bound to a mucus or mucous membrane that adheres to epithelial surfaces (e.g., the gastrointestinal tract, the lung, the eye, etc.), as known to a person skilled in the art. It should point out that mucoadhesive polymers have been widely described in the literature. See, for example, the article entitled “Mucoadhesive Drug Delivery System: A Review” by Dharmendra et al. in Int. J. Pharm. Biol. Arch. 2012, 3(6):1287-1291 and the article entitled “Polymers in Mucoadhesive Drug-Delivery Systes: A Brief Note” published by Roy et al. in Designed Monomers and Polymers 2009, 12(6):483-495.

In general, the invention provides a class of acrylamido monomers each having one (carbamoylphenyl)boronic acid and a pKa of from about 6.4 to about 7.8. It is found that such acrylamido monomers can be prepared from relatively-cheap starting materials according to well-known coupling reactions in the presence of coupling agent.

By having a pKa around a neutral pH (7.0-7.2), an acrylamido monomer of the invention can form a stable cyclo boronate esters with cis-diols. It is advantageously used together with one or more hydrophilic vinylic monomers in preparing vinyl-based copolymers that are mucoadhesive polymers capable of interacting strongly with a mucous or a mucous membrane that adheres to epithelial surfaces. In addition, an acrylamido monomer of the invention has at least two hydrogen bond donors. It is believed that a mucoadhesive polymer containing repeating units of such an acrylamido monomer can have additional interactions with mucin through hydrogen bonding.

In one aspect, the present invention provides an acrylamido monomer of Formula (1)

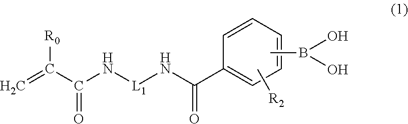

in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.4 to about 7.8 (preferably from about 6.4 to about 7.6). Preferably, the boronic acid is at para position.

An acrylamido monomer of Formula (1) in which L₁ is a C₂-C₆ alkylene divalent radical can be prepared from the following starting materials: (1) an amino-C₂-C₆-alkyl (meth)acrylamide; and (2) a phenylboronic acid compound of Formula (2)

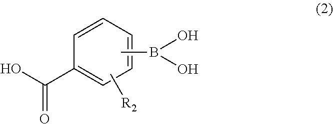

in which R₂ is Cl or NO₂, according to a known coupling reaction between carboxylic acid and amine in the presence of a zero-length coupling agent (e.g., 1-ethyl-3-(3-dimethylaminopropyl) carbodiimide (EDC), N,N′-dicyclohexylcarbodiimide (DCC), 1-cylcohexyl-3-(2-morpholinoethyl)carbodiimide, N,N′-diisopropyl carbodiimide (DIC), or mixtures thereof) and an activating agent (e.g., N-hydroxysuccinimide (NHS)) for forming an amide linkage as known in the art. As an illustrative example, an acrylamido monomer of Formula (1) can be prepared as shown in Scheme I.

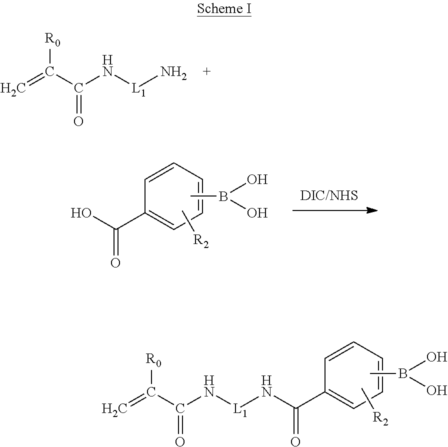

Similarly, one can prepare an acrylamido monomer of Formula (1), in which L₁ is a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, can be prepared from (1) a carboxy-containing (meth)acrylamide or an azlactone-containing vinylic monomer, (2) a phenylboronic acid compound of Formula (2), and (3) an diamine (preferably a mono-Boc-protected diamine), according to the above-described amino-carboxy coupling reaction (to form an amide bond) and ring-opening coupling reaction between azlactone group and amino group —NHR′ (to form an alkylene-diamido linkage of —C(O)NH-CT₄T₅-(CH₂)ₚ—C(O)NR′—).

Examples of preferred phenylboronic acid compounds of Formula (2) include without limitation 4-carboxy-3-chlorophenylboronic acid, 4-carboxy-2-chlorophenylboronic acid, 4-carboxy-3-nitrophenylboronic acid, 4-carboxy-2-nitrophenylboronic acid, 3-carboxy-4-chlorophenylbornic acid, 3-carboxy-5-chlorophenylboronic acid, 3-carboxy-2-chlorophenyl-boronic acid, 3-carboxy-5-nitrophenylboronic acid, 5-carboxy-2-chlorophenylboronic acid, 2-carboxy-4-chlorophenylboronic acid, 2-carboxy-5-chlorophenylboronic acid, and combinations thereof. These preferred phenylboronic acid compounds of Formula (2) can be obtained from commercial sources.

Examples of preferred amino-C₂-C₆-alkyl (meth)acrylamides include without limitation N-(2-aminoethyl) (meth)acrylamide, N-(3-aminopropyl) (meth)acrylamide, N-(2-aminoisopropyl) (meth)acrylamide, N-(4-aminobutyl) (meth)acrylamide, N-(5-aminopentyl) (meth)acrylamide, N-(6-aminohexyl) (meth)acrylamide, and combinations thereof.

Examples of preferred carboxy-containing (meth)acrylamides include without limitation N-(2-carboxypropyl) (meth)acrylamide, N-(3-carboxypropyl) (meth)acrylamide, N-2-acrylamidoglycolic acid, and combinations thereof.

Preferred examples of azlactone-containing vinylic monomers include without limitation 2-vinyl-4,4-dimethyl-1,3-oxazolin-5-one, 2-isopropenyl-4,4-dimethyl-1,3-oxazolin-5-one, 2-vinyl-4-methyl-4-ethyl-1,3-oxazolin-5-one, 2-isopropenyl-4-methyl-4-butyl-1,3-oxazolin-5-one, 2-vinyl-4,4-dibutyl-1,3-oxazolin-5-one, 2-isopropenyl-4-methyl-4-dodecyl-1,3-oxazolin-5-one, 2-isopropenyl-4,4-diphenyl-1,3-oxazolin-5-one, 2-isopropenyl-4,4-pentamethylene-1,3-oxazolin-5-one, 2-isopropenyl-4,4-tetramethylene-1,3-oxazolin-5-one, 2-vinyl-4,4-diethyl-1,3-oxazolin-5-one, 2-vinyl-4-methyl-4-nonyl-1,3-oxazolin-5-one, 2-isopropenyl-4-methyl-4-phenyl-1,3-oxazolin-5-one, 2-isopropenyl-4-methyl-4-benzyl-1,3-oxazolin-5-one, 2-vinyl-4,4-pentamethylene-1,3-oxazolin-5-one, and 2-vinyl-4,4-dimethyl-1,3-oxazolin-6-one (with 2-vinyl-4,4-dimethyl-1,3-oxazolin-5-one (VDMO) and 2-isopropenyl-4,4-dimethyl-1,3-oxazolin-5-one (IPDMO) as most preferred azlactone-containing vinylic monomers).

Examples of preferred diamines include without limitation N,N′-bis(hydroxyethyl)-ethylenediamine, N,N′-dimethylethylenediamine, ethylenediamine, N,N′-dimethyl-1,3-propanediamine, N,N′-diethyl-1,3-propanediamine, propane-1,3-diamine, butane-1,4-diamine, pentane-1,5-diamine, hexamethylenediamine, and combinations thereof.

In a preferred embodiment, in Formula (1), L₁ is a C₂-C₆ alkylene divalent radical. Such a preferred acrylamido monomer can be prepared from cheap starting materials in a one-step coupling reaction. Examples of such preferred acrylamido monomers of Formula (1) include without limitation:

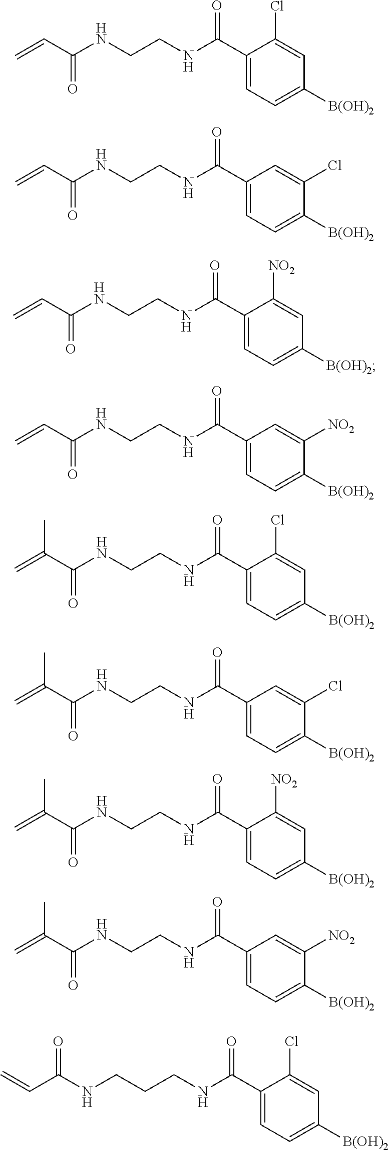

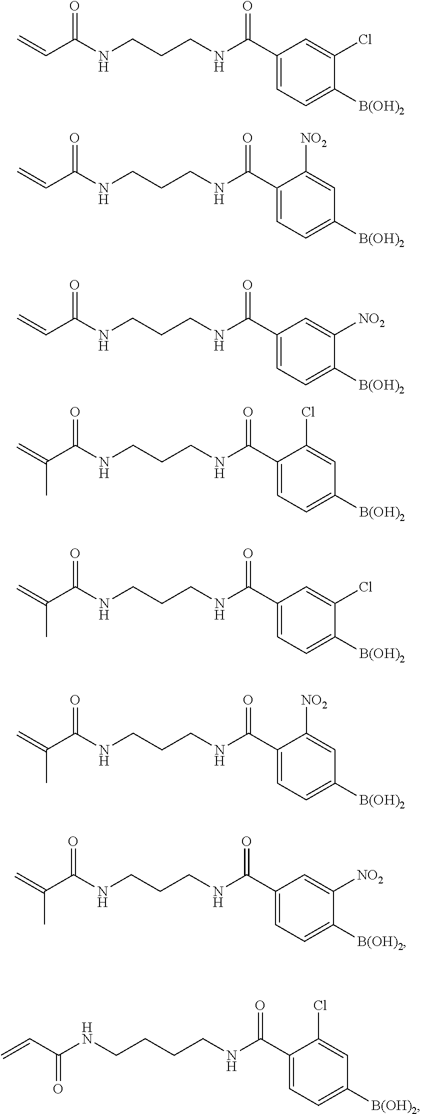

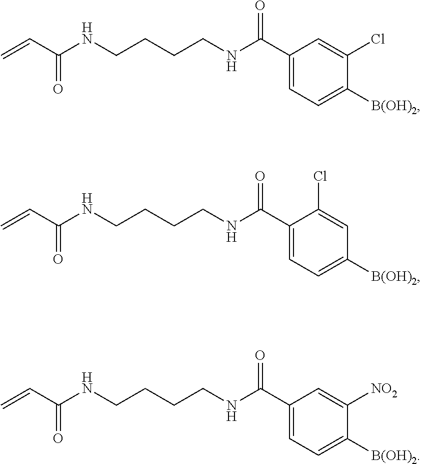 [SMILES: C=CC(=O)NCCCCNC(=O)c1ccc(B(O)O)c(Cl)c1 ; C=CC(=O)NCCCCNC(=O)c1ccc(B(O)O)cc1Cl ; C=CC(=O)NCCCCNC(=O)c1ccc(B(O)O)c([N+](=O)[O-])c1]

An acrylamido monomer of Formula (1) (including those preferred embodiments described above) as defined above can find particular use in preparing a copolymer comprising repeating monomeric units of an acrylamido monomer of Formula (1) and repeating monomeric units of at least one hydrophilic vinylic monomer. Such a copolymer can interact strongly and reversibly with membrane-bound mucins in eye. As such, it can act as an active ingredient/lubricant and mucoadhesive hydrophilic copolymer and also be used in an ophthalmic composition containing demulcens and drugs/comfort agents to prolong the retention of the demulcents and the drugs/comfort agents.

In another aspect, the invention provides a water-soluble hydrophilic copolymer comprising: (a) repeating monomeric units of at least one acrylamido monomer of Formula (1) (as defined above); (b) repeating units of at least one hydrophilic vinylic monomer; and optionally (c) repeating units of at least one hydrophilic vinylic crosslinker, provided that the components (a) and (b) are present in the water-soluble hydrophilic copolymer in a total amount of at least 90% by mole (preferably at least 93% by mole, more preferably at least 96% by mole, even more preferably at least 99% by mole).

In accordance with the invention, a water-soluble hydrophilic copolymer of the invention can be a linear or branched polymer, so long as it can be dissolved in water.

Any suitable hydrophilic vinylic monomers can be used in the preparation of the hydrophilic copolymer of the invention.

Examples of preferred hydrophilic vinylic monomers are alkyl (meth)acrylamides (as described below), hydroxyl-containing acrylic monomers (as described below), amino-containing acrylic monomers (as described below), carboxyl-containing acrylic monomers (as described below), N-vinyl amide monomers (as described below), methylene-containing pyrrolidone monomers (i.e., pyrrolidone derivatives each having a methylene group connected to the pyrrolidone ring at 3- or 5-position) (as described below), acrylic monomers having a C₁-C₄ alkoxyethoxy group (as described below), vinyl ether monomers (as described below), allyl ether monomers (as described below), phosphorylcholine-containing vinylic monomers (as described below), N-2-hydroxyethyl vinyl carbamate, N-carboxyvinyl-β-alanine (VINAL), N-carboxyvinyl-α-alanine, and combinations thereof.

Examples of alkyl (meth)acrylamides include without limitation (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, and combinations thereof.

Examples of hydroxyl-containing acrylic monomers include without limitation N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, 2-hydroxyethyl (meth)acrylate, 3-hydroxypropyl (meth)acrylate, 2-hydroxypropyl (meth)acrylate, di(ethylene glycol) (meth)acrylate, tri(ethylene glycol) (meth)acrylate, tetra(ethylene glycol) (meth)acrylate, poly(ethylene glycol) (meth)acrylate having a number average molecular weight of up to 1500, poly(ethylene glycol)ethyl (meth)acrylamide having a number average molecular weight of up to 1500, and combinations thereof.

Examples of amino-containing acrylic monomers include without limitation N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-aminoethyl (meth)acrylate, 2-methylaminoethyl (meth)acrylate, 2-ethylaminoethyl (meth)acrylate, 3-aminopropyl (meth)acrylate, 3-methylaminopropyl (meth)acrylate, 3-ethylaminopropyl (meth)acrylate, 3-amino-2-hydroxypropyl (meth)acrylate, trimethylammonium 2-hydroxy propyl (meth)acrylate hydrochloride, dimethylaminoethyl (meth)acrylate, and combinations thereof.

Examples of carboxyl-containing acrylic monomers include without limitation 2-(meth)acrylamidoglycolic acid, (meth)acrylic acid, ethylacrylic acid, propylacrylic acid, 3-(meth)acrylamidopropionic acid, 4-(meth)acrylamidobutanoic acid, 5-(meth)acrylamido-pentanoic acid, 3-(meth)acryloyloxypropanoic acid, 4-(meth)acryloyloxybutanoic acid, 5-(meth)acryloyloxypentanoic acid, and combinations thereof.

Examples of preferred N-vinyl amide monomers include without limitation N-vinylpyrrolidone (aka, N-vinyl-2-pyrrolidone), N-vinyl-3-methyl-2-pyrrolidone, N-vinyl-4-methyl-2-pyrrolidone, N-vinyl-5-methyl-2-pyrrolidone, N-vinyl-6-methyl-2-pyrrolidone, N-vinyl-3-ethyl-2-pyrrolidone, N-vinyl-4,5-dimethyl-2-pyrrolidone, N-vinyl-5,5-dimethyl-2-pyrrolidone, N-vinyl-3,3,5-trimethyl-2-pyrrolidone, N-vinyl piperidone (aka, N-vinyl-2-piperidone), N-vinyl-3-methyl-2-piperidone, N-vinyl-4-methyl-2-piperidone, N-vinyl-5-methyl-2-piperidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl caprolactam (aka, N-vinyl-2-caprolactam), N-vinyl-3-methyl-2-caprolactam, N-vinyl-4-methyl-2-caprolactam, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, N-vinyl-N-methyl acetamide, N-vinyl formamide, N-vinyl acetamide, N-vinyl isopropylamide, N-vinyl-N-ethyl acetamide, N-vinyl-N-ethyl formamide, and mixtures thereof. Preferably, the N-vinyl amide monomer is N-vinylpyrrolidone, N-vinyl-N-methyl acetamide, or combinations thereof.

Examples of preferred methylene-containing (═CH₂) pyrrolidone monomers include without limitation 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, and combinations thereof.

Examples of preferred acrylic monomers having a C₁-C₄ alkoxyethoxy group include without limitation ethylene glycol methyl ether (meth)acrylate, di(ethylene glycol) methyl ether (meth)acrylate, tri(ethylene glycol) methyl ether (meth)acrylate, tetra(ethylene glycol) methyl ether (meth)acrylate, C₁-C₄-alkoxy poly(ethylene glycol) (meth)acrylate having a weight average molecular weight of up to 1500 Daltons, methoxy-poly(ethylene glycol)ethyl (meth)acrylamide having a number average molecular weight of up to 1500 Daltons, and combinations thereof.

Examples of preferred vinyl ether monomers include without limitation ethylene glycol monovinyl ether, di(ethylene glycol) monovinyl ether, tri(ethylene glycol) monovinyl ether, tetra(ethylene glycol) monovinyl ether, poly(ethylene glycol) monovinyl ether, ethylene glycol methyl vinyl ether, di(ethylene glycol) methyl vinyl ether, tri(ethylene glycol) methyl vinyl ether, tetra(ethylene glycol) methyl vinyl ether, poly(ethylene glycol) methyl vinyl ether, and combinations thereof.

Examples of preferred allyl ether monomers include without limitation allyl alcohol, ethylene glycol monoallyl ether, di(ethylene glycol) monoallyl ether, tri(ethylene glycol) monoallyl ether, tetra(ethylene glycol) monoallyl ether, poly(ethylene glycol) monoallyl ether, ethylene glycol methyl allyl ether, di(ethylene glycol) methyl allyl ether, tri(ethylene glycol) methyl allyl ether, tetra(ethylene glycol) methyl allyl ether, poly(ethylene glycol) methyl allyl ether, and combinations thereof.

Examples of preferred phosphorylcholine-containing vinylic monomers include without limitation (meth)acryloyloxyethyl phosphorylcholine (aka, MPC, or 2-((meth)acryloyloxy)ethyl-2′-(trimethylammonio)ethylphosphate), (meth)acryloyloxypropyl phosphorylcholine (aka, 3-((meth)acryloyloxy)propyl-2′-(trimethylammonio)ethylphosphate), 4-((meth)acryloyloxy)butyl-2′-(trimethylammonio)ethylphosphate, 2-[(meth)acryloylamino]-ethyl-2′-(trimethylammonio)-ethylphosphate, 3-[(meth)acryloylamino]propyl-2′-(trimethyl-ammonio)ethylphosphate, 4-[(meth)acryloylamino]butyl-2′-(trimethylammonio)ethyl-phosphate, 5-((meth)acryloyloxy)pentyl-2′-(trimethylammonio)ethyl phosphate, 6-((meth)acryloyloxy)hexyl-2-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethyl-phosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)-butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio)ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)-ethyl-2′-(trimethylammonio)ethyl phosphate, 2-(butenoyloxy)ethyl-2′-(trimethylammonio)-ethylphosphate, and combinations thereof.

Any suitable hydrophilic vinylic crosslinkers can be used in the invention. Examples of preferred hydrophilic vinylic crosslinkers include without limitation ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1,000 daltons, glycerol di-(meth)acrylate, glycerol 1,3-diglycerolate di-(meth)acrylate, ethylenebis[oxy(2-hydroxypropane-1,3-diyl)]di-(meth)acrylate, bis[2-(meth)acryloxyethyl]phosphate, diacrylamide (i.e., N-(1-oxo-2-propenyl)-2-propenamide), dimethacrylamide (i.e., N-(1-oxo-2-methyl-2-propenyl)-2-methyl-2-propenamide), N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxy-ethylene bis(meth)acrylamide, N,N′-propylene bis(meth)acrylamide, N,N′-2-hydroxy-propylene bis(meth)acrylamide, N,N′-2,3-dihydroxybutylene bis(meth)acrylamide, 1,3-bis(meth)acrylamide-propane-2-yl dihydrogen phosphate (i.e., N,N′-2-phosphonyloxy-propylene bis(meth)acrylamide), piperazine diacrylamide (or 1,4-bis(meth)acryloyl piperazine), and combinations thereof, more preferably selected from the group consisting of ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1000 Daltons, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, and combinations thereof.

In a preferred embodiment, the water-soluble hydrophilic copolymer of the invention comprises (a) from about 0.1% by mole to about 10% by mole (preferably from about 0.5% to about 8% by mole, more preferably from about 1% to about 6% by mole, even more preferably from about 1% to about 5% by mole) of repeating monomeric units of at least one acrylamido monomer of Formula (1) (as defined above), (b) from about 85% by mole to about 99.9% by mole (preferably from about 90% to about 99.9% by mole, more preferably from about 95% to about 99.9% by mole) of repeating monomeric units of at least one hydrophilic vinylic monomer, and (c) from 0% to about 0.20% by mole (preferably from 0 to about 0.15% by mole, more preferably from 0% to about 0.1% by mole) of repeating monomeric units of at least one hydrophilic vinylic crosslinker, provided that the sum of the mole percentages of components (a), (b), (c), and other components not listed above is 100%. In a preferred embodiment, the acrylamido monomer of Formula (1) has a pKa of from about 6.5 to 7.5, preferably from about 6.8 to about 7.5, even more preferably from about 6.9 to about 7.4.

In another preferred embodiment, the water soluble hydrophilic copolymer of the invention comprises (a) from about 0.1% by mole to about 10% by mole (preferably from about 0.5% to about 8% by mole, more preferably from about 1% to about 6% by mole, even more preferably from about 1% to about 5% by mole) of repeating monomeric units of at least one acrylamido monomer of Formula (1) (as defined above), (b)(1) from about 5% to about 35% by mole (preferably from about 10% to about 30% by mole) of repeating monomeric units of at least one zwitterionic vinylic monomer (preferably at least one phosphorylcholine-containing vinylic monomer), (b)(2) from about 60% to about 94.9% by mole (more preferably from about 65% to about 94% by mole, even more preferably from about 70% to about 93% by mole) of repeating units of at least one hydrophilic vinylic monomer free of any zwitterionic group, and (c) from 0% to about 0.20% by mole (preferably from 0% to about 0.15% by mole, more preferably from 0% to about 0.1% by mole) of repeating monomeric units of at least one hydrophilic vinylic crosslinker, provided that the sum of the mole percentages of components (a), (b)(1), (b)(2), (c) and other components not listed above is 100%. In a preferred embodiment, the acrylamido monomer of Formula (1) has a pKa of from about 6.5 to 7.5, preferably from about 6.8 to about 7.5, even more preferably from about 6.9 to about 7.4.

In accordance with the invention, the mole percentages of each type of repeating monomeric units (i.e., monomeric units) of a water-soluble hydrophilic copolymer of the invention can be determined based on the mole percentage of a vinylic monomer, from which this type of repeating units are derived, in a polymerizable composition for forming the water-soluble hydrophilic copolymer of the invention.

In accordance with the invention, a water-soluble hydrophilic copolymer of the invention has a number average molecular weight of from about 10,000 Daltons to about 5,000,000 Daltons, preferably from about 25,000 Daltons to 2,000,000 Daltons, more preferably from about 50,000 Daltons to about 1,000,000 Daltons.

A person skilled in the art knows well how to prepare a water-soluble hydrophilic copolymer of the invention according to any known polymerization technique. For example, it can be obtained by thermal or actinic polymerization of a polymerizable composition comprising all the required polymerizable components, a free-radical initiator (thermal initiator or photoinitiator), and optionally (but preferably) a chain transfer agent. A chain transfer agent (containing at least one thiol group) is used to control the molecular weight of the resultant copolymer.

The polymerizable composition for preparing a copolymer of the invention can a solution in which all necessary component is dissolved in an inert solvent (i.e., should not interfer with the reaction between the reactants in the mixture), such as water, an organic solvent, or mixture thereof, as known to a person skilled in the art.

Example of suitable solvents include without limitation, water, tetrahydrofuran, tripropylene glycol methyl ether, dipropylene glycol methyl ether, ethylene glycol n-butyl ether, ketones (e.g., acetone, methyl ethyl ketone, etc.), diethylene glycol n-butyl ether, diethylene glycol methyl ether, ethylene glycol phenyl ether, propylene glycol methyl ether, propylene glycol methyl ether acetate, dipropylene glycol methyl ether acetate, propylene glycol n-propyl ether, dipropylene glycol n-propyl ether, tripropylene glycol n-butyl ether, propylene glycol n-butyl ether, dipropylene glycol n-butyl ether, tripropylene glycol n-butyl ether, propylene glycol phenyl ether dipropylene glycol dimetyl ether, polyethylene glycols, polypropylene glycols, ethyl acetate, butyl acetate, amyl acetate, methyl lactate, ethyl lactate, i-propyl lactate, methylene chloride, 2-butanol, 1-propanol, 2-propanol, menthol, cyclohexanol, cyclopentanol and exonorborneol, 2-pentanol, 3-pentanol, 2-hexanol, 3-hexanol, 3-methyl-2-butanol, 2-heptanol, 2-octanol, 2-nonanol, 2-decanol, 3-octanol, norborneol, tert-butanol, tert-amyl, alcohol, 2-methyl-2-pentanol, 2,3-dimethyl-2-butanol, 3-methyl-3-pentanol, 1-methylcyclohexanol, 2-methyl-2-hexanol, 3,7-dimethyl-3-octanol, 1-chloro-2-methyl-2-propanol, 2-methyl-2-heptanol, 2-methyl-2-octanol, 2-methyl-2-nonanol, 2-methyl-2-decanol, 3-methyl-3-hexanol, 3-methyl-3-heptanol, 4-methyl-4-heptanol, 3-methyl-3-octanol, 4-methyl-4-octanol, 3-methyl-3-nonanol, 4-methyl-4-nonanol, 3-methyl-3-octanol, 3-ethyl-3-hexanol, 3-methyl-3-heptanol, 4-ethyl-4-heptanol, 4-propyl-4-heptanol, 4-isopropyl-4-heptanol, 2,4-dimethyl-2-pentanol, 1-methylcyclopentanol, 1-ethylcyclopentanol, 1-ethylcyclopentanol, 3-hydroxy-3-methyl-1-butene, 4-hydroxy-4-methyl-1-cyclopentanol, 2-phenyl-2-propanol, 2-methoxy-2-methyl-2-propanol 2,3,4-trimethyl-3-pentanol, 3,7-dimethyl-3-octanol, 2-phenyl-2-butanol, 2-methyl-1-phenyl-2-propanol and 3-ethyl-3-pentanol, 1-ethoxy-2-propanol, 1-methyl-2-propanol, t-amyl alcohol, isopropanol, 1-methyl-2-pyrrolidone, N,N-dimethylpropionamide, dimethyl formamide, dimethyl acetamide, dimethyl propionamide, N-methyl pyrrolidinone, and mixtures thereof.

The copolymerization of a polymerizable composition for preparing a copolymer of the invention may be induced photochemically or preferably thermally. Suitable thermal polymerization initiators are known to the skilled artisan and comprise, for example peroxides, hydroperoxides, azo-bis(alkyl- or cycloalkylnitriles), persulfates, percarbonates or mixtures thereof. Examples are benzoylperoxide, tert.-butyl peroxide, di-tert.-butyl-diperoxy-phthalate, tert.-butyl hydroperoxide, azo-bis(isobutyronitrile) (AIBN), 1,1-azodiisobutyramidine, 1,1′-azo-bis (1-cyclohexanecarbonitrile), 2,2′-azo-bis(2,4-dimethyl-valeronitrile) and the like. The polymerization is carried out conveniently in an above-mentioned solvent at elevated temperature, for example at a temperature of from 25 to 100° C. and preferably 40 to 80° C. The reaction time may vary within wide limits, but is conveniently, for example, from 1 to 24 hours or preferably from 2 to 12 hours. It is advantageous to previously degas the components and solvents used in the polymerization reaction and to carry out said copolymerization reaction under an inert atmosphere, for example under a nitrogen or argon atmosphere. Copolymerization can yield optical clear well-defined copolymers which may be worked up in conventional manner using for example extraction, precipitation, ultrafiltration and the like techniques.

A water-soluble hydrophilic copolymer of the invention can find particular use in developing ophthalmic compositions for topical application, and in particular artificial tear compositions, which comprise compounds that lubricate and protect the ocular surface. In the context of dry eye disorders, artificial tear compositions can prevent symptoms such as pain and discomfort, can prevent bioadhesion and tissue damage induced by friction, and can encourage the natural healing and restoration of previously damaged tissues.

Ophthalmic compositions are typically developed with a target viscosity to ensure that they are comfortable for the user and do not cause undesirable side effects such as blurring. A suitable viscosity can help ensure that an ophthalmic composition used in dry eye disorders will relieve dry eye-associated symptoms and/or treat the underlying disorder. In drug delivery applications, the viscosity of ophthalmic compositions may be chosen to ensure that a pharmaceutical agent carried in the composition remains in the eye desirably for a longer time.

Natural polymers or chemically-modified derivatives thereof, which display thixotropic and viscoelastic properties, are well-known and have been used as lubricants and ocular surface protectants in many ophthalmic compositions existed in the prior art. Examples of such polymers include hydroxypropyl methylcellulose, galactomannans such as guar and hydroxypropyl guar, carboxymethylcellulose, hyaluronic acid, sodium alginate, polyvinyl alcohol, and the likes. The shear thinning and viscoelastic profiles of those polymers play important roles when mixed with the tear film.

It is believed that a copolymer of the invention can form cyclo boronate ester linkages with 1,2-diol and 1,3-diol moieties existed in those polymers to impart a gel-forming behavior to an ophthalmic composition.

An ophthalmic composition may include one or more additional pharmaceutically active agents. In some embodiments, the one or more pharmaceutically active agents may be selected from the group of phospholipids, ocular lubricants, anti-redness relievers such as brimonidine tartrate, tetrahydrozoline, naphazoline, cooling agents such as menthol, steroids and nonsteroidal anti-inflammatory agents to relieve ocular pain and inflammation, antibiotics, anti-histamines such as olopatadine, anti-virals, antibiotics and anti-bacterials for infectious conjunctivitis, anti-muscarinics such as atropine and derivatives thereof for myopia treatment, and glaucoma drug delivery such as prostaglandin and prostaglandin analogs such as travoprost, or therapeutically suitable combinations thereof.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part or can be combined in any manner and/or used together, as illustrated below:

1. An acrylamido monomer of Formula (1)

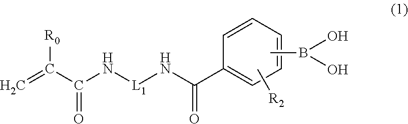

in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.4 to about 7.8.

2. The acrylamido monomer of embodiment 1, wherein in Formula (1) L₁ is a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups.

3. The acrylamido monomer of embodiment 1, wherein in Formula (1) L₁ is a C₂-C₆ alkylene divalent radical.

4. The acrylamido monomer of embodiment 1, being selected from the group consisting of:

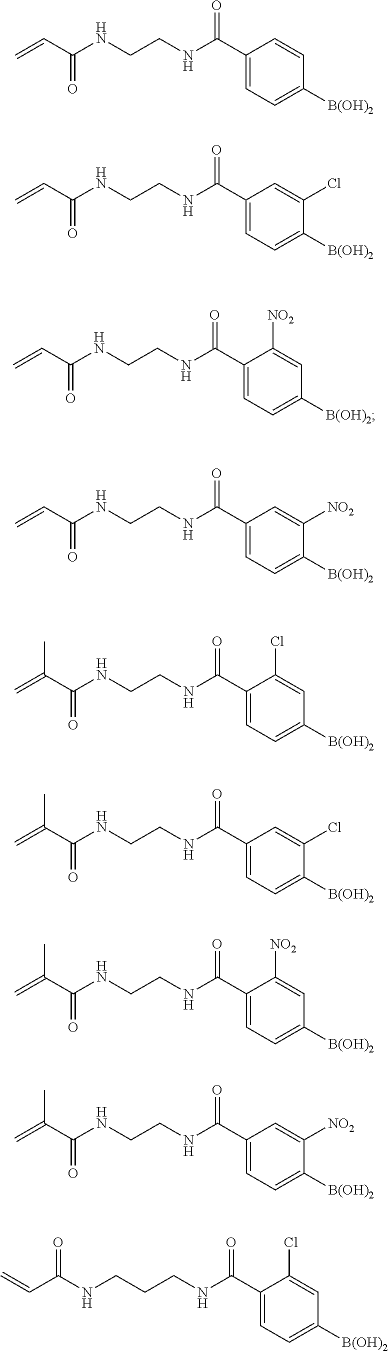

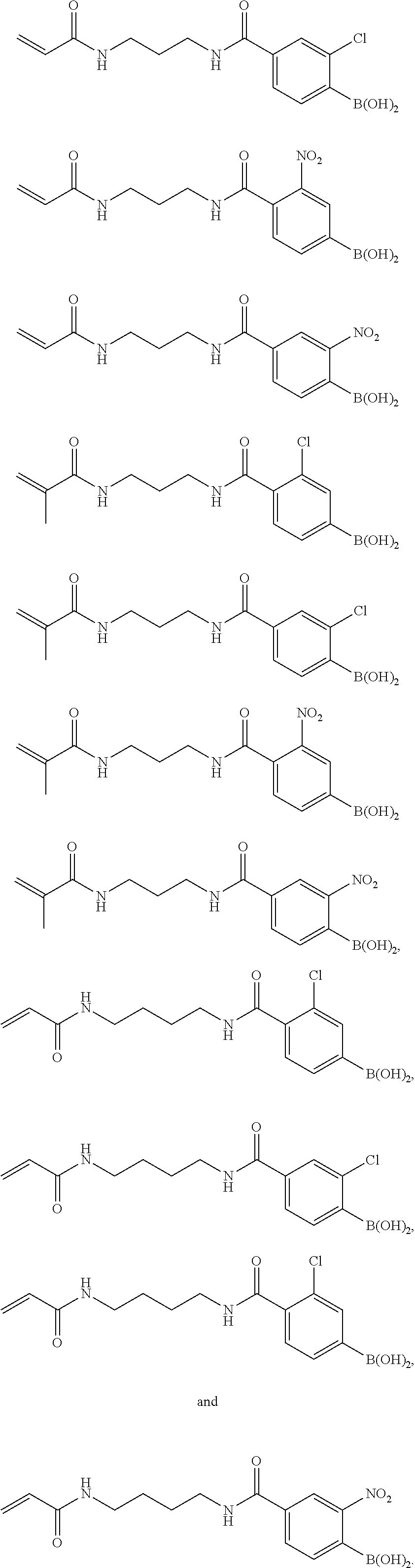

5. The acrylamido monomer of any one of embodiments 1 to 4, wherein the acrylamido monomer has a pKa of from about 6.5 to 7.5.

6. The acrylamido monomer of any one of embodiments 1 to 4, wherein the acrylamido monomer has a pKa of from about 6.8 to about 7.5.

7. The acrylamido monomer of any one of embodiments 1 to 4, wherein the acrylamido monomer has a pKa of from about 6.9 to about 7.4.

8. A water-soluble hydrophilic copolymer, comprising:
(a) repeating monomeric units of at least one acrylamido monomer of any one of embodiments 1 to 7;
(b) repeating units of at least one hydrophilic vinylic monomer; and optionally
(c) repeating units of at least one hydrophilic vinylic crosslinker,
provided that the components (a) and (b) are present in the water-soluble hydrophilic copolymer in a total amount of at least 90% by mole.

9. The water-soluble hydrophilic copolymer of embodiment 8, wherein the components (a) and (b) are present in the water-soluble hydrophilic copolymer in a total amount of at least 93% by mole, more preferably at least 96% by mole, even more preferably at least 99% by mole.

10. The water-soluble hydrophilic copolymer of embodiment 8, wherein the components (a) and (b) are present in the water-soluble hydrophilic copolymer in a total amount of at least 96% by mole.

11. The water-soluble hydrophilic copolymer of embodiment 8, wherein the components (a) and (b) are present in the water-soluble hydrophilic copolymer in a total amount of at least 99% by mole.

12. The water-soluble hydrophilic copolymer of any one of embodiments 8 to 11, wherein the water-soluble hydrophilic copolymer comprises (a) from about 0.1% by mole to about 10% by mole of repeating monomeric units of said at least one acrylamido monomer and (b) from about 85% by mole to about 99.9% by mole of repeating monomeric units of said at least one hydrophilic vinylic monomer.

13. The water-soluble hydrophilic copolymer of embodiment 12, wherein the water-soluble hydrophilic copolymer comprises from about 0.5% to about 8% by mole of repeating monomeric units of said at least one acrylamido monomer.

14. The water-soluble hydrophilic copolymer of embodiment 12, wherein the water-soluble hydrophilic copolymer comprises from about 1% to about 6% by mole of repeating monomeric units of said at least one acrylamido monomer.

15. The water-soluble hydrophilic copolymer of embodiment 12, wherein the water-soluble hydrophilic copolymer comprises from about 1% to about 5% by mole of repeating monomeric units of said at least one acrylamido monomer.

16. The water-soluble hydrophilic copolymer of any one of embodiments 12 to 15, wherein the water-soluble hydrophilic copolymer comprises from about 90% to about 99.9% by mole of repeating monomeric units of said at least one hydrophilic vinylic monomer.

17. The water-soluble hydrophilic copolymer of any one of embodiments 12 to 15, wherein the water-soluble hydrophilic copolymer comprises from about 95% to about 99.9% by mole of repeating monomeric units of said at least one hydrophilic vinylic monomer.

18. The water-soluble hydrophilic copolymer of any one of embodiments 12 to 17, wherein the water-soluble hydrophilic copolymer comprises from 0% to about 0.20% by mole of repeating monomeric units of at least one hydrophilic vinylic crosslinker.

19. The water-soluble hydrophilic copolymer of any one of embodiments 12 to 17, wherein the water-soluble hydrophilic copolymer comprises from 0% to about 0.15% by mole of repeating monomeric units of at least one hydrophilic vinylic crosslinker.

20. The water-soluble hydrophilic copolymer of any one of embodiments 12 to 17, wherein the water-soluble hydrophilic copolymer comprises from 0% to about 0.1% by mole of repeating monomeric units of at least one hydrophilic vinylic crosslinker.

21. The water-soluble hydrophilic copolymer of any one of embodiments 8 to 17, wherein said at least one hydrophilic vinylic monomer comprises (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, 2-hydroxyethyl (meth)acrylate, 3-hydroxypropyl (meth)acrylate, 2-hydroxypropyl (meth)acrylate, di(ethylene glycol) (meth)acrylate, tri(ethylene glycol) (meth)acrylate, tetra(ethylene glycol) (meth)acrylate, poly(ethylene glycol) (meth)acrylate having a number average molecular weight of up to 1500, poly(ethylene glycol)ethyl (meth)acrylamide having a number average molecular weight of up to 1500, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-aminoethyl (meth)acrylate, 2-methylaminoethyl (meth)acrylate, 2-ethylaminoethyl (meth)acrylate, 3-aminopropyl (meth)acrylate, 3-methylaminopropyl (meth)acrylate, 3-ethylaminopropyl (meth)acrylate, 3-amino-2-hydroxypropyl (meth)acrylate, trimethylammonium 2-hydroxy propyl (meth)acrylate hydrochloride, dimethylaminoethyl (meth)acrylate, 2-(meth)acrylamidoglycolic acid, (meth)acrylic acid, ethylacrylic acid, propylacrylic acid, 3-(meth)acrylamidopropionic acid, 4-(meth)acrylamidobutanoic acid, 5-(meth)acrylamidopentanoic acid, 3-(meth)acryloyloxypropanoic acid, 4-(meth)acryloyloxybutanoic acid, 5-(meth)acryloyloxypentanoic acid, N-vinylpyrrolidone, N-vinyl-3-methyl-2-pyrrolidone, N-vinyl-4-methyl-2-pyrrolidone, N-vinyl-5-methyl-2-pyrrolidone, N-vinyl-6-methyl-2-pyrrolidone, N-vinyl-3-ethyl-2-pyrrolidone, N-vinyl-4,5-dimethyl-2-pyrrolidone, N-vinyl-5,5-dimethyl-2-pyrrolidone, N-vinyl-3,3,5-trimethyl-2-pyrrolidone, N-vinyl piperidone, N-vinyl-3-methyl-2-piperidone, N-vinyl-4-methyl-2-piperidone, N-vinyl-5-methyl-2-piperidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl caprolactam, N-vinyl-3-methyl-2-caprolactam, N-vinyl-4-methyl-2-caprolactam, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, N-vinyl-N-methyl acetamide, N-vinyl formamide, N-vinyl acetamide, N-vinyl isopropylamide, N-vinyl-N-ethyl acetamide, N-vinyl-N-ethyl formamide, 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, ethylene glycol methyl ether (meth)acrylate, di(ethylene glycol) methyl ether (meth)acrylate, tri(ethylene glycol) methyl ether (meth)acrylate, tetra(ethylene glycol) methyl ether (meth)acrylate, C₁-C₄-alkoxy poly(ethylene glycol) (meth)acrylate having a weight average molecular weight of up to 1500, methoxy-poly(ethylene glycol)ethyl (meth)acrylamide having a number average molecular weight of up to 1500, ethylene glycol monovinyl ether, di(ethylene glycol) monovinyl ether, tri(ethylene glycol) monovinyl ether, tetra(ethylene glycol) monovinyl ether, poly(ethylene glycol) monovinyl ether, ethylene glycol methyl vinyl ether, di(ethylene glycol) methyl vinyl ether, tri(ethylene glycol) methyl vinyl ether, tetra(ethylene glycol) methyl vinyl ether, poly(ethylene glycol) methyl vinyl ether, allyl alcohol, ethylene glycol monoallyl ether, di(ethylene glycol) monoallyl ether, tri(ethylene glycol) monoallyl ether, tetra(ethylene glycol) monoallyl ether, poly(ethylene glycol) monoallyl ether, ethylene glycol methyl allyl ether, di(ethylene glycol) methyl allyl ether, tri(ethylene glycol) methyl allyl ether, tetra(ethylene glycol) methyl allyl ether, poly(ethylene glycol) methyl allyl ether, a phosphorylcholine-containing vinylic monomer, N-2-hydroxyethyl vinyl carbamate, N-carboxyvinyl-β-alanine (VINAL), N-carboxyvinyl-α-alanine, or combinations thereof.

22. The water-soluble hydrophilic copolymer of any one of embodiments 8 to 21, wherein said at least one hydrophilic vinylic crosslinker comprises ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1,000 daltons, glycerol di-(meth)acrylate, glycerol 1,3-diglycerolate di-(meth)acrylate, ethylenebis[oxy(2-hydroxypropane-1,3-diyl)]di-(meth)acrylate, bis[2-(meth)acryloxyethyl]phosphate, diacrylamide (i.e., N-(1-oxo-2-propenyl)-2-propenamide), dimethacrylamide (i.e., N-(1-oxo-2-methyl-2-propenyl)-2-methyl-2-propenamide), N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamide-propane-2-yl dihydrogen phosphate (i.e., N,N′-2-phosphonyloxypropylene bis(meth)acrylamide), piperazine diacrylamide (or 1,4-bis(meth)acryloyl piperazine), and combinations thereof, more preferably selected from the group consisting of ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1000 Daltons, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, and combinations thereof.

23. The water-soluble hydrophilic copolymer of any one of embodiments 8 to 22, wherein the water-soluble hydrophilic copolymer comprises from about 5% to about 35% by mole of repeating monomeric units of at least one phosphorylcholine-containing vinylic monomer and from about 60% to about 94.9% by mole of repeating units of at least one hydrophilic vinylic monomer other than phosphorylcholine-containing vinylic monomer.

24. The water-soluble hydrophilic copolymer of embodiment 23, wherein the water-soluble hydrophilic copolymer comprises from about 10% to about 30% by mole of repeating monomeric units of at least one phosphorylcholine-containing vinylic monomer.

25. The water-soluble hydrophilic copolymer of embodiment 23 or 24, wherein the water-soluble hydrophilic copolymer comprises from about 65% to about 94% by mole of repeating units of at least one hydrophilic vinylic monomer other than phosphorylcholine-containing vinylic monomer.

26. The water-soluble hydrophilic copolymer of embodiment 23 or 24, wherein the water-soluble hydrophilic copolymer comprises from about 70% to about 93% by mole of repeating units of at least one hydrophilic vinylic monomer other than phosphorylcholine-containing vinylic monomer.

27. The water-soluble hydrophilic copolymer of any one of embodiments 23 to 26, wherein said at least one phosphorylcholine-containing vinylic monomer is selected from the group consisting of (meth)acryloyloxyethyl phosphorylcholine, (meth)acryloyloxypropyl phosphorylcholine, 4-((meth)acryloyloxy)butyl-2′-(trimethylammonio)ethylphosphate, 2-[(meth)acryloylamino]ethyl-2′-(trimethylammonio)-ethylphosphate, 3-[(meth)acryloylamino]propyl-2′-(trimethylammonio)ethylphosphate, 4-[(meth)acryloylamino]butyl-2′-(trimethylammonio)ethylphosphate, 5-((meth)acryloyloxy)pentyl-2′-(trimethylammonio)ethyl phosphate, 6-((meth)acryloyloxy)hexyl-2′-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethylphosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio)ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)ethyl-2′-(trimethylammonio)ethyl phosphate, 2-(butenoyloxy)ethyl-2′-(trimethylammonio)ethylphosphate, and combinations thereof.

28. The water-soluble hydrophilic copolymer of any one of embodiments 8 to 27, wherein said at least one hydrophilic vinylic monomer comprises at least one hydrophilic acrylamido monomer selected from the group consisting of (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, poly(ethylene glycol)ethyl (meth)acrylamide having a number average molecular weight of up to 1500, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-(meth)acrylamidoglycolic acid, 3-(meth)acrylamidopropionic acid, 4-(meth)acrylamidobutanoic acid, 5-(meth)acrylamidopentanoic acid, and combinations thereof.

29. The water-soluble hydrophilic copolymer of any one of embodiments 8 to 27, wherein said at least one hydrophilic vinylic monomer comprises a hydrophilic acrylic monomer selected from the group consisting of 2-hydroxyethyl (meth)acrylate, 3-hydroxypropyl (meth)acrylate, 2-hydroxypropyl (meth)acrylate, di(ethylene glycol) (meth)acrylate, tri(ethylene glycol) (meth)acrylate, tetra(ethylene glycol) (meth)acrylate, poly(ethylene glycol) (meth)acrylate having a number average molecular weight of up to 1500, poly(ethylene glycol)ethyl (meth)acrylamide having a number average molecular weight of up to 1500, 2-aminoethyl (meth)acrylate, 2-methylaminoethyl (meth)acrylate, 2-ethylaminoethyl (meth)acrylate, 3-aminopropyl (meth)acrylate, 3-methylaminopropyl (meth)acrylate, 3-ethylaminopropyl (meth)acrylate, 3-amino-2-hydroxypropyl (meth)acrylate, trimethylammonium 2-hydroxy propyl (meth)acrylate hydrochloride, dimethylaminoethyl (meth)acrylate, (meth)acrylic acid, ethylacrylic acid, propylacrylic acid, 3-(meth)acryloyloxypropanoic acid, 4-(meth)acryloyloxybutanoic acid, 5-(meth)acryloyloxypentanoic acid, and combinations thereof.

30. The water-soluble hydrophilic copolymer of any one of embodiments 8 to 27, wherein said at least one hydrophilic vinylic monomer comprises a vinylic monomer selected from the group consisting of N-vinylpyrrolidone, N-vinyl-N-methyl acetamide, N-vinyl formamide, N-vinyl acetamide, N-vinyl isopropylamide, N-vinyl-N-ethyl acetamide, N-vinyl-N-ethyl formamide, and mixtures thereof.

31. The water-soluble hydrophilic copolymer of any one of embodiments 8 to 30, wherein the water-soluble hydrophilic copolymer has a number average molecular weight of from about 10,000 Daltons to about 10,000,000 Daltons.

32. The water-soluble hydrophilic copolymer of any one of embodiments 1 to 37, wherein the water-soluble hydrophilic copolymer has a number average molecular weight of from about 25,000 Daltons to 5,000,000 Daltons.

33. The water-soluble hydrophilic copolymer of any one of embodiments 1 to 37, wherein the water-soluble hydrophilic copolymer has a number average molecular weight of from about 50,000 Daltons to about 2,000,000 Daltons.

34. An ophthalmic composition comprising: a water-soluble hydrophilic copolymer of any one of embodiments 8 to 33.

The previous disclosure will enable one having ordinary skill in the art to practice the invention. Various modifications, variations, and combinations can be made to the various embodiment described herein. In order to better enable the reader to understand specific embodiments and the advantages thereof, reference to the following examples is suggested. It is intended that the specification and examples be considered as exemplary.

### Abbreviations

The following abbreviations are used in the following examples: CPBA represents (4-(2-acrylamidoethyl)carbamoyl)-3-chlorophenyl)boronic acid; AA represents acrylic acid; APBA represents 3-(acrylamido)phenylboronic acid; MBA represents N,N-methylenebis(acrylamide); MPC represent 2-methacryloyloxyethyl phosphorylcholine; DMA represents N,N-dimethylacrylamide; HEAm represents N-(2-hydroxyethyl)acrylamide; NHS represents N-hydroxysuccinimide; DIC represents diisopropylcarbodiimide; EtOAc represents ethyl acetate; DMF represents dimethylformamide; DIPEA represents N,N-diisopropylethylamine; DMSO represents dimethyl sulfoxide; MeOH represent methanol; mmole represents millimole; TLC represent thin layer chromatograph; CHCl₃ represents chloroform; NMR represents nuclear magnetic resonance; Hz represents hertz; MHz represents megahertz; VAZO 56 represents 2,2′-azobis(2-methylpropionamidine) dihydrochloride; PBS represents a phosphate-buffered saline which has a pH of 7.2±0.2 at 25° C. and contains about 0.044 wt. % NaH₂PO₄·H₂O, about 0.388 wt. % Na₂HPO₄·2H₂O, and about 0.79 wt. % NaCl; DI water represents deionized water; wt. % (% by weight) represents weight percent; w/v % represents a weight/volume percentage concentration (i.e., grams of a solute per 100 mL of solution); min represents minute; MW represents molecular weight; UF represents ultrafiltration; cP represents centipoise; meq represents milliequivalent; MUC2 represent mucin (type Ill) from porcine stomach (from Sigma-Aldrich, catalogue number M1778); DPBS represents Dulbecco's phosphate-buffered saline.

### Example 1

A (4-(2-Acrylamidoethyl)carbamoyl)-3-chlorophenyl)boronic acid (CPBA) is prepared according to the procedure shown in Scheme 1

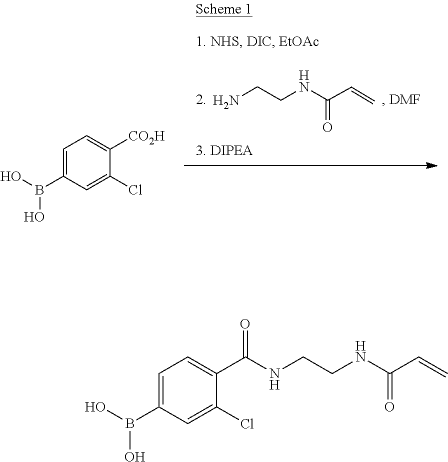

3-3-chloro-4-carboxyphenylboronic acid (0.51 g, 2.52 mmol) and N-Hydroxysuccinimide, NHS (0.32 g, 2.78 mmol) are charged into a 40 mL vial equipped with magnetic stirrer and rubber septum. Ethyl Acetate (15 mL) is added through a septum via syringe. The content of the vial is cooled down to 0° C. in ice-water bath. Diisopropyl-carbodiimide, DIC (0.35 g, 2.77 mmol) dissolved in 2 mL of ethyl acetate is added dropwise to the cooled solution of 3-chloro-4-carboxyphenylboronic acid and NHS in ethyl acetate. Ice-water bath is removed after 30 min and reaction mixture is allowed to stir overnight at room temperature. Urea precipitate which is formed is filtered off and ethyl acetate is evaporated. The active ester is redissolved in DMF (10 mL) and 2-(aminoethyl)-acrylamide×HCl (0.45 g, 2.99 mmol) is added and stirred for 10 min until all solid dissolves. The content of the reaction flask is cooled down to 0° C. in ice-water bath. N,N-Diisopropyl-ethylamine, DIPEA (0.93 g, 7.20 mmol) combined with 2 mL of DMF is added dropwise to the vial containing NHS-ester and 2-(aminoethyl)acrylamide×HCl. Ice-water bath is removed after 10 min and reaction mixture is allowed to stir for 5 h at room temperature. TLC shows consumption of NHS-ester. DMF is removed on high vacuum pump overnight and crude reaction mixture is purified by silica gel column chromatography (1.5 in×12 in; CHCl₃→CHCl₃:MeOH 100→90:10). Solvent is removed by rotary evaporation and final product is purified by crystallization from MeOH/water mixture. Crystallization over 3 days at room temperature produced white solid which is collected by filtration on Büchner funnel. It is transferred to vial and dried by oil pump vacuum overnight to give 0.372 g (1.25 mmol, 50% yield) of the final product as a white solid.

¹H-NMR (600 MHz, DMSO-d₆+D₂O): δ=8.45 (s, 1H), 8.18 (s, 1H), 7.81 (s, 1H), 7.73 (d, 2H, J=8.0 Hz), 7.41 (d, 2H, J=8.0 Hz), 6.20 (dd, 1H, J=17.0, 10.0 Hz), 6.09 (d, 1H, J=17.0 Hz), 5.60 (d, 1H, J=10.0 Hz), 3.48 (s, 4H).

### Example 2

In current Systane products the mechanism of action relies on the formation of hydroxypropyl-guar (HP-Guar) gel with borate to prolong the retention of demulcents, such as polyethylene glycol and propylene glycol, on the eye. This helps to protect the ocular surface, thereby reducing the symptoms of the dry eye disease. The challenge is that the current retention time of demulcents on eye is not long enough as patients would like to experience.

It is believed that a water-soluble hydrophilic copolymer of the invention can be crosslinked with mucin presented on membrane bound mucins, primarily through reversible cyclo-boronate-ester linkages each formed between one substituted (carbamoylphenyl)boronic acid of the hydrophilic copolymer and one of the cis-diol moieties of the mucin and secondarily through non-covalent interactions such as hydrogen bonds formed between the hydrophilic copolymer and the mucin. As such, a water-soluble hydrophilic copolymer of the invention can act as active ingredients/lubricants and will effectively bind to membrane bound mucins.

It is also believed that when one solution of a water-soluble hydrophilic polymer of the invention is mixed with one solution of a mucin on the eye are mixed, the interactions between a water-soluble hydrophilic polymer of the invention and mucin presented on membrane bound mucins can yield an enhanced viscosity relative to the sum of the viscosities of the two solutions. Such an enhanced viscosity can be a quantitative measure of the polymer-mucin interactions and is designated in this application as a polymer-mucin-interaction synergy.

The mucin selected for the evaluation of interactions between a water-soluble hydrophilic copolymer and mucin is porcine mucin due to its similarity to human eye mucin. The mucin from porcine stomach, type Ill, is ordered from Sigma-Aldrich, catalogue number M1778. The received mucin is purified by ultrafiltration before it is used for this polymer-mucin interaction evaluation.

In this application, the polymer-mucin-interaction synergy (designated as PMIS) of a water-soluble hydrophilic copolymer of the invention is calculated according to Eq. (1)

$$P M I S \% = \frac{{μ}_{mix} - [ {μ}_{hc} + {μ}_{mucin} ]}{[ {μ}_{hc} + {μ}_{mucin} ]} \qquad ( 1 )$$

in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type Ill from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type Ill from porcine stomach in phosphate-buffered saline.

### Assays

MUC2 stock solution (2 wt %) is prepared by dissolving freeze-dried MUC2 in DPBS using speed-mixer (1500-2000 RPM/10 min/2-3 times). A stock solution (2 wt %) of a water-soluble hydrophilic copolymer of the invention is prepared by reconstituting and homogenizing the water-soluble hydrophilic copolymer in in DPBS using speed-mixer (1500-2000 RPM/10 min/2-3 times).

1.2 wt % MUC2 solution is prepared from 2 wt % MUC2 stock solution by dilution with DPBS.

Equal volume (1 mL) of 1.2 wt % MUC2 solution and DPBS are mixed in 20 mL scintillation vial to form a 0.6 wt % MUC2 solution. The prepared 0.6 wt % MUC2 solution is tested for viscosity according to the procedures described below.

Equal volume (1 mL) of 2 wt % hydrophilic copolymer solution and DPBS are mixed in 20 mL scintillation vial to form a 1 wt % copolymer solution. The prepared 1 wt % copolymer solution is tested for viscosity according to the procedures described below.

2 mL of 2.0% copolymer solution is added to a 20 mL scintillation vial with a stir bar. Vial is placed on stir plate set at 500-600 RPM. 2 mL of 1.2% MUC2 solution is added dropwise over the span of 10-20 seconds. Check the mixed solution which does show free flow as a homogenous viscous solution without cluster or coagulated gel. Solution is mixed at 500-600 RPM for 30 min, then mixing speed is reduced to 150-200 RPM for 20-30 min to dissipate bubbles.

Viscosity measurements are carried out in a DVNext Wells-Brookfield Cone/Plate Rheometer as follows. About 0.5 mL of a sample is placed into the sample cup. All viscosities are measured with size 40 spindle at room temperature.

### Example 3

### Copolymer Synthesis

A stock solution of CPBA (4 wt %) is prepared by dissolving a desired amount of CPBA in DMSO; stock solution of Vazo-56 (1 wt %), MBA (1 wt %), and MPC (17 wt %) are prepared by dissolving a desired amount of Vazo-56, MBA or MPC in D.I. water.

Combine the stock solutions with the quantities which meet the target amounts as shown in Table 1, directly in a jacketed reactor equipped with an overhead stirrer, a condenser, thermocouple, and a nitrogen gas dispersion fritted tube. Then, fill the remaining part with water to make a final 5% solid solution ([Vazo-56]=2,2′-azobis(2-methylpropionamidine) dihydrochloride).

TABLE 1

| | | Composition, wt % (% by mole) | | | | | | | | | |
|---|---|---|---|---|---|---|---|---|---|---|---|
| | | 3-1 | | 3-2 | | 3-3 | | 3-4 | 3-5 | | 3-6 |
| Monomer | MW | P(DMA-APBA) | | P(DMA-CPBA) | | P(DMA-AA-CPBA) | | P(HEAm-CPBA) | P(DMA-MPC-CPBA) | | P(HEAm-MPC-CPBA) |
| DMA | 99.1 | 89.9 | (94.5) | 86.2 | (94.5) | 83.9 | (84.0) | — | 49.5 | (74.5) | — |

| | | | | | | | | | |
|---|---|---|---|---|---|---|---|---|---|
| HEAm | 113.1 | — | — | — | 87.0 | (94.5) | — | 53.2 | (74.5) |

| | | | | | | | | | |
|---|---|---|---|---|---|---|---|---|---|
| MPC | 295.3 | — | — | — | — | 39.6 | (20.0) | 36.7 | (20.0) |

| | | | | | | | | |
|---|---|---|---|---|---|---|---|---|
| APBA | 191.0 | 10.1 | (5.5) | — | — | — | — | — |

| | | | | | | | | | | | | |
|---|---|---|---|---|---|---|---|---|---|---|---|---|
| CPBA | 296.5 | — | 14.8 | (5.5) | 5.9 | (2.0) | 13.0 | (5.5) | 10.9 | (5.5) | 10.1 | (5.5) |

| | | |
|---|---|---|
| AA | 10.2 | (14.0) |

| | | | | | | | | | | |
|---|---|---|---|---|---|---|---|---|---|---|
| MBA | 154.2 | — | — | — | 0.06 | (0.06) | 0.12 | (0.12) | 0.12 | (0.12) |

First value represents wt %, values in parentheses represent mole %.

Purge a polymerizable composition with nitrogen to remove oxygen. Left the frit of the gas dispersion tube above the polymerizable composition and the maintain a nitrogen flow around 50 mL/min throughout the polymerization. Ramp the polymerizable composition's temperature from 16° C. to 56° C. over 2 hours. After 10 hours, lower the temperature from 56° C. to 20° C. over 2 hours. Purify the crude polymer solution by ultrafiltration using a 1 M Dalton cut-off membrane to remove the residual monomers, low MW polymers, and any residual solvent. Freeze-dry purified polymer for further characterization and testing.

The purified polymers are characterized. The results are reported in Table 2.

TABLE 2

| | Copolymer Characterization | | | | | |
|---|---|---|---|---|---|---|
| | 3-1 | 3-2 | 3-3 | 3-4 | 3-5 | 3-6 |
| | P(DMA-APBA) | P(DMA-CPBA) | P(DMA-AA-CPBA) | P(HEAm-CPBA) | P(DMA-MPC-CPBA) | P(HEAm-MPC-CPBA) |
| Yield_{UF w/1M} | 98% | 94% | 87% | 96% | 74% | 88% |
| Viscosity | 27 | 136 | 62 | 51 | 15 | 23 |
| (cP)_{0.5% in PBS} | | | | | | |
| FGC_{meq/g} * | 0.51 (_{0.52}) | 0.55 (_{0.50}) | TBD | 0.45 (_{0.44}) | 0.42 (_{0.37}) | 0.39 (_{0.34}) |
| M_{w} × 10⁶ | TBD | TBD | TBD | TBD | TBD | TBD |
| (g/mol)_{D} | | | | | | |

* the content of phenylboronic acid; and the values in parenthesis are the theoretical values based on the composition of the polymerizable composition.

### Example 4

Water-soluble hydrophilic copolymers are prepared according to the procedures described in Example 3 and characterized. The composition of the polymerizable compositions for preparing copolymers and characterization results are reported in Table 3.

TABLE 3

| | 3-5 | | 4-1 | | 4-2 | | 4-3 | |
|---|---|---|---|---|---|---|---|---|
| Monomer | P(DMA-MPC-CPBA) | | P(DMA-MPC-CPBA) | | P(DMA-MPC-CPBA) | | P(DMA-MPC-CPBA) | |
| DMA | 49.5% | (74.5%) | 54.3% | (78.0%) | 56.6% | (79.5%) | 57.2% | (79.95%) |
| MPC | 39.6% | (20%) | 41.5% | (20.0%) | 42.4% | (20.0%) | 42.7% | (20%) |
| CPBA | 10.9% | (5.5%) | 4.20% | (4.16%) | 1.00% | (0.5%) | 0.10% | (0.05%) |

| | | | | | |
|---|---|---|---|---|---|
| MBA | 0.12% | (0.12%) | — | — | — |

Basic Copolymer Properties

| | | | | |
|---|---|---|---|---|
| Yield_{UF w/1M} | 74% | 90% | 85% | 92% |
| Viscosity | 15 | 22 | 39 | 35 |
| (cP)_{0.5% in PBS} | | | | |
| FGC_{meq/g} | 0.42_{0.36} | 0.21_{0.14} | 0.14_{0.04} | n/a |
| M_{w} × 10⁶ | TBD | TBD | TBD | TBD |
| (g/mol)_{D} | | | | |

First value represents wt %, values in parentheses represent mole %.

### Example 5

Water-soluble hydrophilic copolymers are prepared according to the procedures described in Example 3 and characterized. The composition of the polymerizable compositions for preparing copolymers and characterization results are reported in Table 4.

TABLE 4

| | 3-6 | | 5-1 | | 5-2 | | 5-3 | |
|---|---|---|---|---|---|---|---|---|
| Monomer | P(HEAm-MPC-CPBA) | | P(HEAm-MPC-CPBA) | | P(HEAm-MPC-CPBA) | | P(HEAm-MPC-CPBA) | |
| HEAm | 53.2% | (74.5%) | 58.0% | (78.0%) | 60.2% | (79.5%) | 60.9% | (79.95%) |
| MPC | 36.7% | (20%) | 38.2% | (20.0%) | 38.8% | (20.0%) | 39.0% | (20.0%) |
| CPBA | 10.1% | (5.5%) | 3.80% | (2.0%) | 1.00% | (0.5%) | 0.10% | (0.05%) |
| MBA | 0.12% | (0.12%) | 0.06% | (0.06%) | 0.12% | (0.12%) | 0.12% | (0.12%) |

Basic Copolymer Properties

| | | | | |
|---|---|---|---|---|
| Yield_{UF w/1M} | 88% | 91% | 91% | 88% |
| Viscosity | 23 | 26 | 36 | 62 |
| (cP)_{0.5% in PBS} | | | | |
| FGC_{meq/g} | 0.39_{0.34} | 0.26_{0.13} | 0.14_{0.03} | 0.11_{0.003} |
| M_{w} * 10⁶ | TBD | TBD | TBD | TBD |
| (g/mol)_{D} | | | | |

First value represents wt %, values in parentheses represent mole %.

### Example 6

Water-soluble hydrophilic copolymers are prepared according to the procedures described in Example 3 and characterized. The composition of the polymerizable compositions for preparing copolymers and characterization results are reported in Table 5.

TABLE 5

| | 6-1 | | 6-2 | | 6-3 | | 6-4 | |
|---|---|---|---|---|---|---|---|---|
| Monomer | P(HEAm-MPC-CPBA) | | P(HEAm-MPC-CPBA) | | P(HEAm-MPC-CPBA) | | P(HEAm-MPC-CPBA) | |
| HEAm | 74.1% | (88.0%) | 58.0% | (78.0%) | 58.0% | (78.0%) | 45.3% | (68.0%) |
| MPC | 21.5% | (10.0%) | 38.2% | (20.0%) | 38.2% | (20.0%) | 51.2% | (30.0%) |
| CPBA | 4.4% | (2.0%) | 3.8% | (2.0%) | 3.8% | (2.0%) | 3.5% | (2.0%) |
| MBA | 0.06% | (0.06%) | 0.06% | (0.06%) | 0.06% | (0.06%) | 0.06% | (0.06%) |

Basic Copolymer Properties

| | | | | |
|---|---|---|---|---|
| Yield_{UF w/1M} | 90% | 93% | 93% | 93% |
| Viscosity | 47 | 24 | 20 | 26 |
| (cP)_{0.5% in PBS} | | | | |
| FGC_{meq/g} | 0.22_{0.14} | 0.19_{0.15} | 0.22_{0.15} | 0.20_{0.12} |
| M_{w} × 10⁶ | TBD | TBD | TBD | TBD |
| (g/mol)_{D} | | | | |

First value represents wt %, values in parentheses represent mole %.

### Example 7

The polymer-mucin-interaction synergies (PMISs) of the water-soluble hydrophilic copolymers prepared in Example 3 are determined according to the procedures described in Example 2. The results are reported in Tables 6 and 7.

TABLE 6

Copolymer (mole %)

| | 3-1 | 3-2 |
|---|---|---|
| | P(DMA-APBA) | P(DMA-CPBA) |
| DMA | 94.5 | 94.5 |
| APBA | 5.5 | — |
| CPBA | — | 5.5 |
| 7Theoretical Mixed | 7.80 | 8.75 |
| Viscosity (cP) | | |
| Measured | 9.14 | 46.02 |
| Viscosity (cP) | | |
| PMIS (%) | 17% | 426% |

TABLE 7

| | 3-2 | 3-4 | 3-5 | 3-6 |
|---|---|---|---|---|
| | P(DMA-CPBA) | P(HEAm-CPBA) | P(DMA-MPC-CPBA) | P(HEAm-MPC-CPBA) |
| DMA (mole %) | 94.5 | — | 74.5 | — |
| HEAm (mol %) | — | 94.5 | | 74.5 |
| MPC (mole %) | — | — | 20 | 20 |
| CPBA (mole %) | 5.5 | 5.5 | 5.5 | 5.5 |
| Theoretical Mixed | 8.75 | 7.66 | 9.85 | 7.57 |
| Viscosity (cP) | | | | |
| Measured | 46.02 | 140.80 | 33.57 | 42.97 |
| Viscosity (cP) | | | | |
| PMIS (%) | 426% | 1739% | 241% | 468% |

Table 6 shows the comparison of the strength of polymer-mucin interactions of a copolymer of the invention (having repeating monomeric units of CPBA with a pKa of about 7.2) to that of a control copolymer (having repeating monomeric units of APBA with a pKa of about 8.1). A water-soluble hydrophilic copolymer of the invention has stronger mucin interactions than the control copolymer.

Table 7 shows that replacing DMA (N_{H-bond donner}−N_{H-bond acceptor}=0−1=−1) in the polymer backbone with HEAm (N_{H-bond donner}−N_{H-bond acceptor}=2−2=0) significantly enhances polymer-mucin interactions. Viscosity increases from 46 cP (DMA) to 141 cP (HEAm), which translates an PMIS and 426% and 1739% respectively. This result may be hydrogen bonding between HEAm-containing copolymer and mucin due to the presence of H-bond donors in HEAm. On the other hand, addition of MPC (20 mol %) to either copolymer reduces PMIS, i.e., polymer-mucin interactions (Table 7). 2-Methacryloyloxyethyl phosphorylcholine (MPC) may attenuate the mucin interaction, but HEAm seems help to regain the interaction strength.

### Example 8

The polymer-mucin-interaction synergies (PMISs) of the water-soluble hydrophilic copolymers prepared in Example 4 are determined according to the procedures described in Example 2. The results are reported in Table 8.

TABLE 8

| | 3-5 | 4-1 | 4-2 | 4-3 |
|---|---|---|---|---|
| DMA (mole %) | 74.5 | 78 | 79.5 | 79.95 |
| MPC (mole %) | 20 | 20 | 20 | 20 |
| CPBA (mole %) | 5.5 | 2.0 | 0.50 | 0.05 |
| Theoretical Mixed | 10.50 | 8.17 | 9.56 | 7.81 |
| Viscosity (cP) | | | | |
| Measured | 34.66 | 13.24 | 12.33 | 11.27 |
| Viscosity (cP) | | | | |
| PMIS (%) | 230% | 62% | 29% | 35% |

Table 8 shows the effects of the content of the repeating monomeric units of CPBA on PMIS (polymer-mucin interaction strength) of poly(DMA-MPC-CPBA). The higher the CPBA content, the higher the PMIS (i.e., the stronger the polymer-mucin interactions).

### Example 9

The polymer-mucin-interaction synergies (PMISs) of the water-soluble hydrophilic copolymers prepared in Example 5 are determined according to the procedures described in Example 2. The results are reported in Table 9.

TABLE 9

| | 3-6 | 5-1 | 5-2 | 5-3 |
|---|---|---|---|---|
| HEAm (mole %) | 74.5 | 78 | 79.5 | 79.95 |
| MPC (mole %) | 20 | 20 | 20 | 20 |
| CPBA (mole %) | 5.5 | 2.0 | 0.50 | 0.05 |
| MBA (mole %) | 0.12 | 0.06 | 0.12 | 0.12 |
| Theoretical Mixed | 8.63 | 8.43 | 10.55 | 13.75 |
| Viscosity (cP) | | | | |
| Measured | 47.42 | 14.59 | 15.43 | 18.35 |
| Viscosity (cP) | | | | |
| PMIS (%) | 450% | 73% | 46% | 34% |

Table 9 shows the effects of the content of the repeating monomeric units of CPBA on PMIS (polymer-mucin interaction strength) of poly(HEAm-MPC-CPBA). The higher the CPBA content, the higher the PMIS (i.e., the stronger the polymer-mucin interactions).

### Example 10

The polymer-mucin-interaction synergies (PMISs) of the water-soluble hydrophilic copolymers prepared in Example 6 are determined according to the procedures described in Example 2. The results are reported in Table 10.

TABLE 10

| | 6-1 | 6-2 | 6-3 | 6-4 |
|---|---|---|---|---|
| HEAm (mole %) | 88 | 78 | 78 | 68 |
| MPC (mole %) | 10 | 20 | 20 | 30 |
| CPBA (mole %) | 2.0 | 2.0 | 2.0 | 2.0 |
| MBA (mole %) | 0.06 | 0.06 | 0.06 | 0.06 |
| Theoretical Mixed | 12 | 8 | 9 | 8 |
| Viscosity (cP) | | | | |
| Measured | 22.90 | 13.46 | 14.96 | 11.41 |
| Viscosity (cP) | | | | |
| PMIS (%) | 94% | 61% | 71% | 51% |

Table 10 shows the effects of the content of the repeating monomeric units of MPC on PMIS (polymer-mucin interaction strength) of poly(HEAm-MPC-CPBA). The higher the MPC content, the lower the PMIS (i.e., the stronger the polymer-mucin interactions).

All the publications, patents, and patent application publications, which have been cited herein above in this application, are hereby incorporated by reference in their entireties.

What is claimed is:

1. An acrylamido monomer of Formula (1) 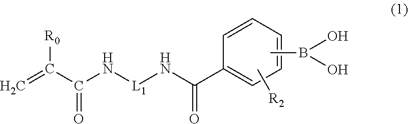
in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.4 to about 7.8.

2. The acrylamido monomer of claim 1, wherein in Formula (1) L₁ is a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups.

3. The acrylamido monomer of claim 1, wherein in Formula (1) L₁ is a C₂-C₆ alkylene divalent radical.

4. The acrylamido monomer of claim 1, being selected from the group consisting of:

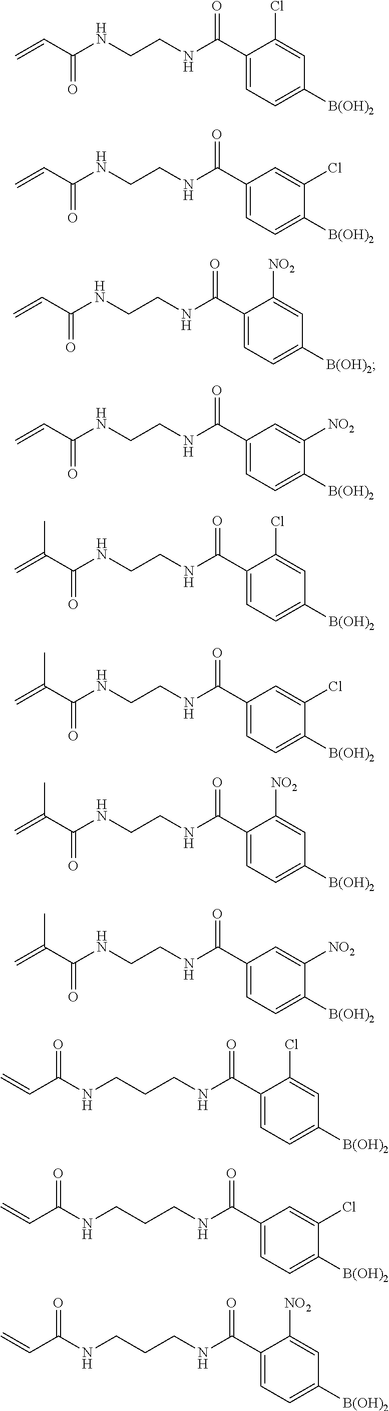

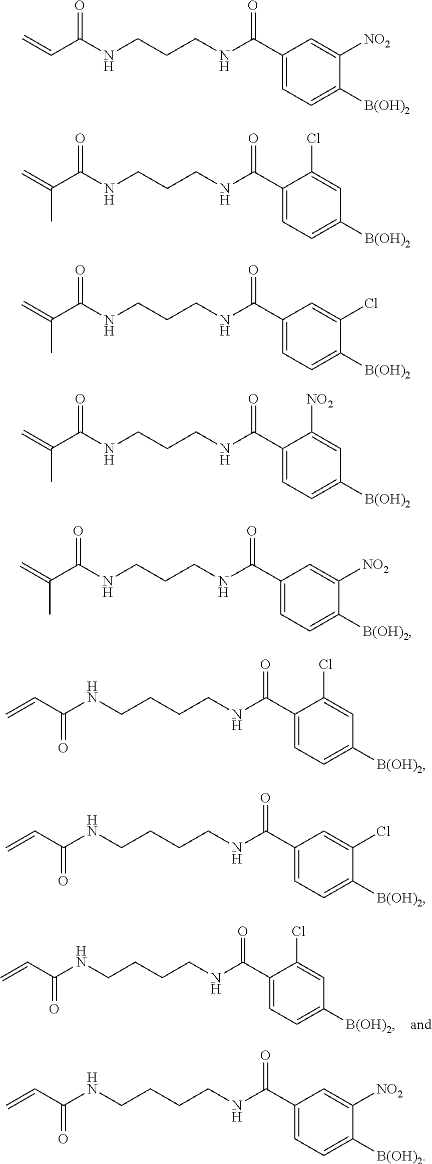

5. The acrylamido monomer of claim 1, wherein the acrylamido monomer has a pKa of from about 6.5 to 7.5.

6. A water-soluble hydrophilic copolymer, comprising:
(a) from about 0.1% by mole to about 10% by mole of repeating monomeric units of repeating monomeric units of at least one acrylamido monomer of Formula (1) 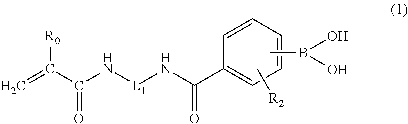
in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.5 to about 7.5;
(b) from about 85% by mole to about 99.9% by mole of repeating units of at least one hydrophilic vinylic monomer; and optionally
(c) from 0% to about 0.20% by mole of repeating units of at least one hydrophilic vinylic crosslinker,
provided that the components (a) and (b) are present in the water-soluble hydrophilic copolymer in a total amount of at least 90% by mole.

7. The water-soluble hydrophilic copolymer of claim 6, wherein said at least one hydrophilic vinylic monomer comprises (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, 2-hydroxyethyl (meth)acrylate, 3-hydroxypropyl (meth)acrylate, 2-hydroxypropyl (meth)acrylate, di(ethylene glycol) (meth)acrylate, tri(ethylene glycol) (meth)acrylate, tetra(ethylene glycol) (meth)acrylate, poly(ethylene glycol) (meth)acrylate having a number average molecular weight of up to 1500, poly(ethylene glycol)ethyl (meth)acrylamide having a number average molecular weight of up to 1500, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-aminoethyl (meth)acrylate, 2-methylaminoethyl (meth)acrylate, 2-ethylaminoethyl (meth)acrylate, 3-aminopropyl (meth)acrylate, 3-methylaminopropyl (meth)acrylate, 3-ethylaminopropyl (meth)acrylate, 3-amino-2-hydroxypropyl (meth)acrylate, trimethylammonium 2-hydroxy propyl (meth)acrylate hydrochloride, dimethylaminoethyl (meth)acrylate, 2-(meth)acrylamidoglycolic acid, (meth)acrylic acid, ethylacrylic acid, propylacrylic acid, 3-(meth)acrylamidopropionic acid, 4-(meth)acrylamidobutanoic acid, 5-(meth)acrylamidopentanoic acid, 3-(meth)acryloyloxypropanoic acid, 4-(meth)acryloyloxybutanoic acid, 5-(meth)acryloyloxypentanoic acid, N-vinylpyrrolidone, N-vinyl-3-methyl-2-pyrrolidone, N-vinyl-4-methyl-2-pyrrolidone, N-vinyl-5-methyl-2-pyrrolidone, N-vinyl-6-methyl-2-pyrrolidone, N-vinyl-3-ethyl-2-pyrrolidone, N-vinyl-4,5-dimethyl-2-pyrrolidone, N-vinyl-5,5-dimethyl-2-pyrrolidone, N-vinyl-3,3,5-trimethyl-2-pyrrolidone, N-vinyl piperidone, N-vinyl-3-methyl-2-piperidone, N-vinyl-4-methyl-2-piperidone, N-vinyl-5-methyl-2-piperidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl caprolactam, N-vinyl-3-methyl-2-caprolactam, N-vinyl-4-methyl-2-caprolactam, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, N-vinyl-N-methyl acetamide, N-vinyl formamide, N-vinyl acetamide, N-vinyl isopropylamide, N-vinyl-N-ethyl acetamide, N-vinyl-N-ethyl formamide, 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, ethylene glycol methyl ether (meth)acrylate, di(ethylene glycol) methyl ether (meth)acrylate, tri(ethylene glycol) methyl ether (meth)acrylate, tetra(ethylene glycol) methyl ether (meth)acrylate, C₁-C₄-alkoxy poly(ethylene glycol) (meth)acrylate having a weight average molecular weight of up to 1500, methoxy-poly(ethylene glycol)ethyl (meth)acrylamide having a number average molecular weight of up to 1500, ethylene glycol monovinyl ether, di(ethylene glycol) monovinyl ether, tri(ethylene glycol) monovinyl ether, tetra(ethylene glycol) monovinyl ether, poly(ethylene glycol) monovinyl ether, ethylene glycol methyl vinyl ether, di(ethylene glycol) methyl vinyl ether, tri(ethylene glycol) methyl vinyl ether, tetra(ethylene glycol) methyl vinyl ether, poly(ethylene glycol) methyl vinyl ether, allyl alcohol, ethylene glycol monoallyl ether, di(ethylene glycol) monoallyl ether, tri(ethylene glycol) monoallyl ether, tetra(ethylene glycol) monoallyl ether, poly(ethylene glycol) monoallyl ether, ethylene glycol methyl allyl ether, di(ethylene glycol) methyl allyl ether, tri(ethylene glycol) methyl allyl ether, tetra(ethylene glycol) methyl allyl ether, poly(ethylene glycol) methyl allyl ether, a phosphorylcholine-containing vinylic monomer, N-2-hydroxyethyl vinyl carbamate, N-carboxyvinyl-β-alanine (VINAL), N-carboxyvinyl-α-alanine, or combinations thereof.

8. The water-soluble hydrophilic copolymer of claim 7, wherein said at least one hydrophilic vinylic crosslinker comprises ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1,000 daltons, glycerol di-(meth)acrylate, glycerol 1,3-diglycerolate di-(meth)acrylate, ethylenebis[oxy(2-hydroxypropane-1,3-diyl)]di-(meth)acrylate, bis[2-(meth)acryloxyethyl]phosphate, diacrylamide (i.e., N-(1-oxo-2-propenyl)-2-propenamide), dimethacrylamide (i.e., N-(1-oxo-2-methyl-2-propenyl)-2-methyl-2-propenamide), N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamide-propane-2-yl dihydrogen phosphate (i.e., N,N′-2-phosphonyloxypropylene bis(meth)acrylamide), piperazine diacrylamide (or 1,4-bis(meth)acryloyl piperazine), and combinations thereof.

9. The water-soluble hydrophilic copolymer of claim 8, wherein the water-soluble hydrophilic copolymer comprises from about 5% to about 35% by mole of repeating monomeric units of at least one phosphorylcholine-containing vinylic monomer and from about 60% to about 94.9% by mole of repeating units of at least one hydrophilic vinylic monomer other than phosphorylcholine-containing vinylic monomer.

10. The water-soluble hydrophilic copolymer of claim 9, wherein the water-soluble hydrophilic copolymer comprises from about 65% to about 94% by mole of repeating units of at least one hydrophilic vinylic monomer other than phosphorylcholine-containing vinylic monomer.

11. The water-soluble hydrophilic copolymer of claim 10, wherein said at least one phosphorylcholine-containing vinylic monomer is selected from the group consisting of (meth)acryloyloxyethyl phosphorylcholine, (meth)acryloyloxypropyl phosphorylcholine, 4-((meth)acryloyloxy)butyl-2′-(trimethylammonio)ethylphosphate, 2-[(meth)acryloylamino]ethyl-2′-(trimethylammonio)-ethylphosphate, 3-[(meth)acryloylamino]propyl-2′-(trimethylammonio)ethylphosphate, 4-[(meth)acryloylamino]butyl-2′-(trimethylammonio)ethylphosphate, 5-((meth)acryloyloxy)pentyl-2′-(trimethylammonio)ethyl phosphate, 6-((meth)acryloyloxy)hexyl-2′-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethylphosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio)ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)ethyl-2′-(trimethylammonio)ethyl phosphate, 2-(butenoyloxy)ethyl-2′-(trimethylammonio)ethylphosphate, and combinations thereof.

12. The water-soluble hydrophilic copolymer of claim 11, wherein said at least one hydrophilic vinylic monomer comprises at least one hydrophilic acrylamido monomer selected from the group consisting of (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, poly(ethylene glycol)ethyl (meth)acrylamide having a number average molecular weight of up to 1500, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-(meth)acrylamidoglycolic acid, 3-(meth)acrylamidopropionic acid, 4-(meth)acrylamidobutanoic acid, 5-(meth)acrylamidopentanoic acid, and combinations thereof.

13. The water-soluble hydrophilic copolymer of claim 11, wherein said at least one hydrophilic vinylic monomer comprises a hydrophilic acrylic monomer selected from the group consisting of 2-hydroxyethyl (meth)acrylate, 3-hydroxypropyl (meth)acrylate, 2-hydroxypropyl (meth)acrylate, di(ethylene glycol) (meth)acrylate, tri(ethylene glycol) (meth)acrylate, tetra(ethylene glycol) (meth)acrylate, poly(ethylene glycol) (meth)acrylate having a number average molecular weight of up to 1500, poly(ethylene glycol)ethyl (meth)acrylamide having a number average molecular weight of up to 1500, 2-aminoethyl (meth)acrylate, 2-methylaminoethyl (meth)acrylate, 2-ethylaminoethyl (meth)acrylate, 3-aminopropyl (meth)acrylate, 3-methylaminopropyl (meth)acrylate, 3-ethylaminopropyl (meth)acrylate, 3-amino-2-hydroxypropyl (meth)acrylate, trimethylammonium 2-hydroxy propyl (meth)acrylate hydrochloride, dimethylaminoethyl (meth)acrylate, (meth)acrylic acid, ethylacrylic acid, propylacrylic acid, 3-(meth)acryloyloxypropanoic acid, 4-(meth)acryloyloxybutanoic acid, 5-(meth)acryloyloxypentanoic acid, and combinations thereof.

14. The water-soluble hydrophilic copolymer of claim 11, wherein said at least one hydrophilic vinylic monomer comprises a vinylic monomer selected from the group consisting of N-vinylpyrrolidone, N-vinyl-N-methyl acetamide, N-vinyl formamide, N-vinyl acetamide, N-vinyl isopropylamide, N-vinyl-N-ethyl acetamide, N-vinyl-N-ethyl formamide, and mixtures thereof.

15. The water-soluble hydrophilic copolymer of claim 11, wherein the water-soluble hydrophilic copolymer has a number average molecular weight of from about 10,000 Daltons to about 10,000,000 Daltons.

16. An ophthalmic composition comprising a water-soluble hydrophilic copolymer of a water-soluble hydrophilic copolymer which comprises:
(a) from about 0.1% by mole to about 10% by mole of repeating monomeric units of repeating monomeric units of at least one acrylamido monomer of Formula (1) 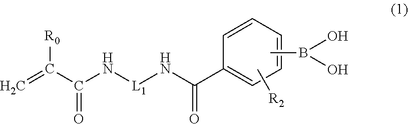
in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.5 to about 7.5;
(b) from about 85% by mole to about 99.9% by mole of repeating units of at least one hydrophilic vinylic monomer; and optionally
(c) from 0% to about 0.20% by mole of repeating units of at least one hydrophilic vinylic crosslinker,
provided that the components (a) and (b) are present in the water-soluble hydrophilic copolymer in a total amount of at least 90% by mole.

17. The ophthalmic composition of claim 16, wherein the water-soluble hydrophilic copolymer comprises from about 5% to about 35% by mole of repeating monomeric units of at least one phosphorylcholine-containing vinylic monomer and from about 60% to about 94.9% by mole of repeating units of at least one hydrophilic vinylic monomer other than phosphorylcholine-containing vinylic monomer.

18. The ophthalmic composition of claim 17, wherein the water-soluble hydrophilic copolymer comprises from about 65% to about 94% by mole of repeating units of at least one hydrophilic vinylic monomer other than phosphorylcholine-containing vinylic monomer.

19. The ophthalmic composition of claim 18, wherein said at least one hydrophilic vinylic monomer comprises at least one hydrophilic acrylamido monomer selected from the group consisting of (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, poly(ethylene glycol)ethyl (meth)acrylamide having a number average molecular weight of up to 1500, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, 2-(meth)acrylamidoglycolic acid, 3-(meth)acrylamidopropionic acid, 4-(meth)acrylamidobutanoic acid, 5-(meth)acrylamidopentanoic acid, and combinations thereof.
